(19) United States
(12) Patent Application Publication
(10) Pub. No.: US 2023/0225711 A1
(43) Pub. Date: Jul. 20, 2023

(54) SYSTEMS AND METHODS FOR AUTOMATICALLY DETERMINING AND DISPLAYING A VASCULAR DEPTH MEASUREMENT ON AN ULTRASOUND IMAGE DISPLAYED ON A DEVICE

(71) Applicant: Clarius Mobile Health Corp., Vancouver (CA)

(72) Inventor: Kris Dickie, Vancouver (CA)

(21) Appl. No.: 18/097,467

(22) Filed: Jan. 16, 2023

Related U.S. Application Data
(60) Provisional application No. 63/300,157, filed on Jan. 17, 2022.

(51) Int. Cl. A61B 8/08; A61B 8/00

(52) U.S. Cl. CPC A61B 8/5223; A61B 8/463; A61B 8/465; A61B 8/488; A61B 8/5207

(57) ABSTRACT

A method for automatically determining a depth of a vascular feature on an ultrasound image feed, acquired from an ultrasound scanner comprises displaying, on a screen that is communicatively connected to the ultrasound scanner, the ultrasound image feed comprising ultrasound image frames of a region of interest comprising the vascular feature, activating a Doppler mode of the ultrasound scanner, in which the ultrasound scanner obtains a Doppler-mode ultrasound signal corresponding to the region of interest comprising the vascular feature, applying at least one image processing filter to preserve the Doppler-mode ultrasound signal (the “preserved Doppler-mode signal”), generating from the preserved Doppler-mode signal of the vascular feature as returned to the ultrasound scanner, the depth of the vascular feature and indicating depth of the vascular feature to a user of ultrasound scanner

### FIELD OF THE INVENTION

The present disclosure relates generally to ultrasound imaging, and in particular, systems and methods for performing a measurement on an ultrasound image displayed on a device.

### BACKGROUND OF THE INVENTION

Ultrasound imaging systems are a powerful tool for performing real-time imaging procedures in a wide range of medical applications. For example, in intervention procedures (e.g., nerve blocks, vascular access), needles are often used for administration of medicine or evacuation of fluid contents. Also, a variety of aesthetic and cosmetic procedures are based around injectables. Ultrasound allows for high-resolution imaging of the skin from the stratum corneum down to the deep fascia. With fast acquisition times, ultrasound images are produced in real-time, allowing for physical adjustments and optimal imaging. Many devices include color Doppler analysis for characterization of blood flow and vessel morphology all of which can be incorporated into clinical practise.

In the U.S. over 13 million non-invasive procedures were performed on patients, including dermal filler injections and injections of neurotoxins such as Botox®, Dysport® and Xeomin®. Operators and clinicians are acutely of the complications that can arise during injectable and filler administration if a vascular structure is inadvertently targeted and filled with the injectable material. Filler that enters a blood vessel can cause skin necrosis (death of tissue), direct vessel occlusion, stroke, or blindness. These resulting complications can be serious, permanent, and possibly fatal.

To assist professionals who are providing cosmetic and aesthetic procedures to patients/clients, ultrasound scanning is used to located vascular structures and to determine the depth of those structures from the dermis. Currently available techniques to determine vascular depth assessment involve multiple steps during Doppler imaging, including acquiring image, freezing a selected image, placing a first edge of a caliper on an upper portion of a vascular feature, placing the second edge of the caliper on or near the dermis. Problems can be compounded by using a touchscreen to precisely place the edges of the calipers since a fingertip of an ultrasound operator may typically be larger than that of the arrowhead of a cursor manipulated by manual controls (e.g., a trackball). These challenges may be even more pronounced in instances where the ultrasound operator is wearing protective gloves as they have less tactile feedback about finger placement.

Additionally, the ultrasound operator/professional must take time away from the procedure at hand (ex: cosmetic or aesthetic procedure) in order to carry through each step of this workflow.

There is thus a need for improved ultrasound systems and methods for performing a measurement of the depth of a vascular feature in an ultrasound image. The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

Non-limiting examples of various embodiments of the present disclosure will next be described in relation to the drawings, in which:

FIG. 1A is a flowchart diagram showing steps of a method of automatically performing a depth measurement on vascular feature on an ultrasound image displayed on a device, in accordance with at least one embodiment of the present invention;

FIG. 1B is another flowchart diagram showing steps of a method of automatically performing a depth measurement on vascular feature on an ultrasound image displayed on a device, in accordance with at least one embodiment of the present invention;

FIG. 2A is an image showing the result of performing acts of the present methods on an example ultrasound image, in accordance with at least one embodiment of the present invention;

FIG. 2B is an image showing the result of performing acts of the present methods on an example ultrasound image, in accordance with at least one embodiment of the present invention;

FIG. 2C is an image showing the result of performing acts of the present methods on an example ultrasound image, in accordance with at least one embodiment of the present invention;

FIG. 2D is an image showing the result of performing acts of the present methods on an example ultrasound image, in accordance with at least one embodiment of the present invention;

FIG. 3A is an image showing the result of performing acts of the present methods on an example ultrasound image, in accordance with at least one embodiment of the present invention;

FIG. 3B is an image showing the result of performing acts of the present methods on an example ultrasound image, in accordance with at least one embodiment of the present invention;

FIG. 3C is an image showing the result of performing acts of the present methods on an example ultrasound image, in accordance with at least one embodiment of the present invention; and

FIG. 4 shows a functional block diagram of an ultrasound system, in accordance with at least one embodiment of the present invention.

Unless otherwise specifically noted, articles depicted in the drawings are not necessarily drawn to scale.

### DETAILED DESCRIPTION OF THE INVENTION

### A. Glossary

The term “communications network” and “network” can include both a mobile network and data network without limiting the term’s meaning, and includes the use of wireless (e.g. 2G, 3G, 4G, 5G, WiFi®, WiMAX®, Wireless USB (Universal Serial Bus), Zigbee®, Bluetooth® and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “depth” when relating to an ultrasound image refers to a measure of how far a vascular feature (or features) being scanned is from a skin surface (upon which is transducer is placed and employed) wherein such a measurement is highly instructive for an operator/medical practitioner for the purposes of therapy, procedures, diagnosis, or treatment.

The term “module” can refer to any component in this invention and to any or all of the features of the invention without limitation. A module may be a software, firmware or hardware module (or part thereof), and may be located or operated within, for example, in the ultrasound scanner, a display device or a server.

The term “multi-purpose electronic device” or “display device” is intended to have broad meaning and includes devices with a processor communicatively operable with a screen interface, for example, such as, laptop computer, a tablet computer, a desktop computer, a smart phone, a smart watch, spectacles with a built-in display, a television, a bespoke display or any other display device that is capable of being communicably connected to an ultrasound scanner. Such a device may be communicatively operable with an ultrasound scanner and/or a cloud-based server (for example via one or more communications networks).

The term “operator” (or “user”) may (without limitation) refer to the person that is operating an ultrasound scanner (for example, a clinician, medical personnel, aesthetics professional, dentist, a sonographer, ultrasound student, ultrasonographer and/or ultrasound technician).

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as in cloud computing or local virtualization, which are ultimately grounded in physical processors.

The term “system” when used herein, and not otherwise qualified, refers to a system for enabling an automatic depth measurement of one or more vascular features on an ultrasound image feed and indicating such depth to an operator/user. In various embodiments, the system may include an ultrasound scanner and a display device; and/or an ultrasound scanner, display device and a server.

The term “ultrasound image frame” (or “image frame” or “ultrasound frame”) refers to a frame of either pre-scan data or post-scan conversion data that is suitable for rendering an ultrasound image on a screen or other display device.

The term “ultrasound transducer” (or “probe” or “ultrasound probe” or “transducer” or “ultrasound scanner” or “scanner”) refers to a wide variety of transducer types including but not limited to linear transducer, curved transducers, curvilinear transducers, convex transducers, microconvex transducers, and endocavity probes.

Thee term “vascular feature” as used herein and to which the depth measurement method, system and tool of the invention may be applied, (for example, the methods, processes and systems described herein), is, broadly and without limitation, any vascular feature and tissue through which blood flows and for which a depth analysis (for example from a patient skin surface, upon which scanner is placed and employed) is desired, for any therapy, procedure, diagnosis, or treatment. As such, “vascular feature” comprises (but is not limited to) arteries which include, but are not limited to the group consisting of carotid artery, subclavian artery, axillary artery, brachial artery, radial artery, ulnar artery, aorta, hypergastic artery, external iliac artery, femoral artery, popliteal artery, anterior tibial artery, arteria dorsalis celiac artery, cystic artery, common hepatic artery (hepatic artery proper, gastric duodenal artery, right gastric artery), right gastroepiploic artery, superior pancreaticoduodenal artery, inferior pancreaticoduodenal artery, pedis artery, posterior tibial artery, ophthalmic artery facial artery, angular artery, superficial temporal artery, superior labial artery, inferior labial artery, and retinal artery.

### B. Exemplary Embodiments

In a first broad aspect of the present disclosure, there are provided ultrasound systems and ultrasound-based methods for automatically measuring the depth of a vascular feature, within a region of interest (ROI) by processing a Doppler signal, such signal being an indicator of depth.

In another aspect of the present disclosure, there is provided a method for measuring the depth of a vascular feature which comprises receiving a Doppler signal of an image displayed on a screen during ultrasound scanning, said image comprising a vascular feature within a region of interest, using the Doppler signal to automatically calculate in real-time and without additional user inputs, a depth value of the vascular feature, and indicating the depth value on an interface accessible by a user. In some embodiments the depth value is stored.

In another aspect of the present disclosure there is provided a method for automatically determining a depth of a vascular feature on an ultrasound image feed, acquired from an ultrasound scanner, the method comprising: displaying, on a screen that is communicatively connected to the ultrasound scanner, the ultrasound image feed comprising ultrasound image frames of a region of interest comprising the vascular feature; activating a Doppler mode of the ultrasound scanner, in which the ultrasound scanner obtains a Doppler-mode ultrasound signal corresponding to the region of interest comprising the vascular feature; applying at least one image processing filter to preserve the Doppler-mode ultrasound signal (the “preserved Doppler-mode signal”); generating from the preserved Doppler-mode signal of the vascular feature as returned to the ultrasound scanner, the depth of the vascular feature; and indicating depth of the vascular feature to a user of ultrasound scanner.

In another aspect of the present disclosure there is provided an ultrasound imaging system for automatically determining a depth of a vascular feature on an ultrasound image feed, comprising: an ultrasound scanner configured to acquire a plurality of new ultrasound frames; processor that is communicatively connected to the ultrasound scanner and configured to: display, on a screen that is communicatively connected to the ultrasound scanner, the ultrasound image feed comprising ultrasound image frames of a region of interest comprising the vascular feature; activate a Doppler mode of the ultrasound scanner, in which the ultrasound scanner obtains a Doppler-mode ultrasound signal corresponding to the region of interest comprising the vascular feature; apply at least one image processing filter to preserve the Doppler-mode ultrasound signal (the “preserved Doppler-mode signal”); generate from the preserved Doppler-mode signal of the vascular feature as returned to the ultrasound scanner, the depth of the vascular feature; and a display device configured to indicate the depth of the vascular feature to a system user.

In another aspect of the present disclosure there is provided a computer readable medium storing instruction for execution by a processor communicatively coupled with an ultrasound scanner, within an ultrasound imaging system, wherein when the instructions are executed by the processor, it is configured to: display, on a screen that is communicatively connected to the ultrasound scanner, an ultrasound image feed comprising ultrasound image frames of a region of interest comprising a vascular feature; activate a Doppler mode of the ultrasound scanner, in which the ultrasound scanner obtains a Doppler-mode ultrasound signal corresponding to the region of interest comprising the vascular feature; apply at least one image processing filter to preserve the Doppler-mode ultrasound signal (the “preserved Doppler-mode signal”); generate from the preserved Doppler-mode signal of the vascular feature as returned to the ultrasound scanner, the depth of the vascular feature; indicate depth of the vascular feature to a user of the ultrasound imaging system.

In another aspect of the present disclosure, there is provided another system for use in measuring the depth of a vascular feature which comprises an ultrasound scanner and a touchscreen device capable of communicating with the ultrasound scanner, the touchscreen device includes: a processor; and a memory storing instructions for execution by the processor, the interface user trigger for initiating an automated depth measurement on the vascular feature within an ultrasound image displayed on a touchscreen device, wherein when the instructions are executed by the processor, the processor is configured to: i) receive, via the touchscreen device, direction to measure depth of a vascular feature by receiving inputs of a Doppler signal of an image displayed on a screen during ultrasound scanning, said image comprising the vascular feature within a region of interest, ii) use the Doppler signal to automatically calculate in real-time and without additional user inputs, a depth value of the vascular feature; and iii) display the depth value on a touchscreen interface viewable by a user.

In another aspect of the present disclosure, there is provided a computer readable medium storing instructions for performing an automatic depth measurement of a vascular feature within a region of interest on an ultrasound image displayed on a touchscreen device, the instructions for execution by a processor of a touchscreen device, wherein when the instructions are executed by the processor, the processor is configured to: i) receive, via the touchscreen device, direction to measure depth of a vascular feature by receiving inputs of a Doppler signal of an image displayed on a screen during ultrasound scanning, said image comprising the vascular feature within a region of interest, ii) use the Doppler signal to automatically calculate in real-time and without additional user inputs, a depth value of the vascular feature; and iii) indicate the depth value in a manner accessible by a user (for example, viewable on touchscreen or audible signal or message).

In another aspect of the present disclosure, there is provided a touchscreen device which is capable of communicating with an ultrasound scanner, the touchscreen device includes: a processor; and a memory storing instructions for execution by the processor, a interface user trigger for initiating an automated depth measurement on a vascular feature within an ultrasound image displayed on a touchscreen device, wherein when the instructions are executed by the processor, the processor is configured to: i) receive, via the touchscreen device, direction to measure depth of a vascular feature using inputs of a Doppler signal of an image displayed on a screen during ultrasound scanning, said image comprising the vascular feature within a region of interest, ii) determine automatically, in real-time and without additional user inputs, a depth value of the vascular feature using the Doppler signal; and iii) indicate the depth value in a manner accessible by a user (for example, viewable on touchscreen or audible signal or message).

In another aspect of the present disclosure, there is provided a method for measuring the depth of a vascular feature which comprises: i) receive a first signal which comprises a plurality of B-mode images of a vascular feature within a region of interest; ii) optimize one or more parameters of the B-mode images; iii) convert first signal to a Doppler mode signal thereby displaying Doppler mode images; iv) increase/optimize persistence of Doppler mode images; v) use the Doppler mode signal to automatically calculate in real-time and without additional user inputs, a depth value of the vascular feature; and vi) indicate the depth value in a manner accessible by a user (for example, viewable on touchscreen or audible signal or message). In some embodiments the depth value is also stored.

In another aspect of the present disclosure, there is provided a workflow tool for measuring the depth of a vascular feature, enabled by an operator of an ultrasound scanner, in real-time and while scanning, without additional any manual caliper movements. Along with these workflows, the present invention comprises the underlying graphical user interface organized to deploy the method of the invention, including a user selection “flow depth indicator” option. This workflow tool may be implemented through an ultrasound scanner, or through a multi-use device communicatively associated with an ultrasound scanner or through an application operated though a cloud-based server communicatively associated with one or both of an ultrasound scanner and a multi-use device. A graphical user interface organized to deploy the method of the invention may be viewable on a screen, for example a touchscreen, on a multi-use device communicatively associated with an ultrasound scanner.

The system of the present invention uses a transducer (a piezoelectric or capacitive device operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer.

Ultrasound imaging systems may generally be operated in various Doppler modes that take advantage of the fact that reflected echoes undergo a change in frequency when reflected by moving objects in tissue (e.g., blood in vascular tissue). Some Doppler modes include spectral Doppler, pulsed wave (PW) Doppler, continuous wave (CW) Doppler, color Doppler, and Power Doppler. Tissue Doppler Imaging (TDI) is also a particular way of using spectral or Color Doppler for visualizing tissue wall motion using a lower frequency signal acquisition rate. It can be interchanged with the use of Power Doppler and Color Doppler as necessary.

Color Doppler produces a color-coded map of Doppler shifts superimposed onto a B-mode ultrasound image. Blood flow direction depends on whether the motion is toward or away from the transducer. Selected by convention, red and blue colors provide information about the direction and velocity of the blood flow i.e., red is accepted to mean there is flow towards the ultrasound probe and blue is accepted to mean that there is flow away from the ultrasound probe, unless these colors are inverted.

When an ultrasound scanner is used in a power Doppler mode, it allows the operator to select a specific, small area on the image, and, in the tissue corresponding to that area, measure blood motion velocity. As part of this process, a gate is specified by the user, along an ultrasound beam line or direction (e.g., a one-dimensional signal is obtained). Color doppler provides information about the presence or absence of flow, mean flow velocity and direction of flow within a selected color box on an anatomical feature. Spectral Doppler differs from Color Doppler imaging in that information is not obtained from the entire color box (as placed) but from a specified gate window, as noted above, a generally 2-4 mm wide sample volume. In power Doppler the magnitude of the color flow output is displayed rather than the Doppler frequency signal. Power Doppler does not display flow direction or different velocities, so it is often used in conjunction with frame averaging to increase sensitivity to low flows and velocities.

Color flow Doppler ultrasound produces a color-coded map of Doppler shifts superimposed onto a B-mode ultrasound image (color flow maps). Although color flow imaging uses pulsed wave ultrasound, its processing differs from that used to provide the Doppler sonogram. Color flow imaging may have to produce several thousand color points of flow information for each frame superimposed on the B-mode image. Color flow imaging uses fewer, shorter pulses along each color scan line of the image to give a mean frequency shift and a variance at each small area of measurement. This frequency shift is displayed as a color pixel. The scanner then repeats this for several lines to build up the color image, which is superimposed onto the B-mode image. The transducer elements are switched rapidly between B-mode and color flow imaging to give an impression of a combined simultaneous image. The pulses used for color flow imaging are typically three to four times longer than those for the B-mode image, with a corresponding loss of axial resolution. Assignment of color to frequency shifts is usually based on direction (for example, red for Doppler shifts towards the ultrasound beam and blue for shifts away from it) and magnitude (different color hues or lighter saturation for higher frequency shifts). The color Doppler image is dependent on general Doppler factors, particularly the need for a good beam/flow angle. Curvilinear and phased array transducers have a radiating pattern of ultrasound beams that can produce complex color flow images, depending on the orientation of the arteries and veins. In practice, the experienced operator alters the scanning approach to obtain good insonation angles so as to achieve unambiguous flow images.

Color Doppler ultrasound uses the same principles as pulsed wave Doppler. Within a region of interest (ROI, for example a color Doppler box) many different “sample volumes or pixel areas” are assessed the velocity and direction of flow (for each individual area) is calculated. This information is then encoded in color according to a color map scheme (which can be chosen by the operator) and displayed for each imaging frame (dynamic color flow imaging).

Within the scope of one preferred aspect of the invention, a vascular feature may be automatically, and without additional user intervention, identified and a depth of a vascular feature determined, by a depth analysis module, which receives and analyzes Doppler-mode signals of the vascular feature, as returned to the ultrasound scanner. Prior to implementing the depth analysis module of the present invention, a preserved Doppler-mode signal of the vascular feature is created (“preserved Doppler signal”) by applying at least one image processing filter to preserve the Doppler-mode ultrasound signal.

Within the scope of the invention, any Doppler signal may be used to determine depth of the vascular feature, but Power Doppler and Color Doppler are most preferred. Power Doppler is even more sensitive than color Doppler in detecting blood flow (for examples in areas of lesser flow and smaller vessels such as in facial medical aesthetic applications) but does not provide information about the direction of blood flow, which is not generally necessary within the scope of the invention.

Within the scope of the invention, one or more temporal filters are applied to preserve the Doppler-mode ultrasound signals of the vascular feature. In one aspect, this is achieved by increasing/optimizing the persistence/frame averaging of Doppler mode images and temporal filtering is applied prior to a depth measurement act/applying the depth analysis module. Temporal resolution is described by a frame rate which is defined as the number of ultrasound images displayed in one second and is expressed in Hertz (Hz). High frame rates enable viewing of rapidly moving structures (such as heart valves) without motion artifacts, and also perform velocity and deformation analysis (i.e., tissue Doppler). Persistence refers to temporal smoothing used in both gray scale and color Doppler imaging. Successive frames are averaged as they are displayed to reduce the variations in the image between frames, hence lowering the temporal resolution of the image. Adjusting the image persistence causes individual frames of the scan to linger, thus blending them with the images in the successive frames. This causes incremental degrees of smoothing to the ultrasound image. Increasing persistence will smooth the image and reduce the frame rate; however, it can also create ghosting. Persistence can be increased within the scope of the present invention to preserve the Doppler-mode ultrasound signals of the vascular feature a higher level than would be generally acceptable from a diagnostic perspective as the end goal of the present invention is simply to quickly and accurately identify the vascular feature so that the depth analysis module can be applied thereto. Likewise, increasing the number of frames to a higher level than would be generally acceptable from a diagnostic perspective, preserves the Doppler-mode ultrasound signals of the vascular feature so that the depth analysis module can be applied thereto. In creating a preserved Doppler signal, a more accurate determination may be made of a vascular feature’s depth in the subsequent analysis module (i.e., reading the depth of the returned signal, from the preserved Doppler signal). As the end goal of the method and system of the invention is not an analysis of blood flow, a analysis of the condition of the vessels or other core diagnostic steps, increasing either or both of persistence and frame rate to a higher level than conventionally used, and the generally accepted negative issues related to such increases, in order to created the preserved Doppler signal, is not detrimental but rather aids in determining: i) a more highly confirmed location of vascular feature and ii) the depth of such vascular feature.

There are a variety of techniques known and employed in the art to apply temporal filters to ultrasound images. Without limiting the generality of the foregoing, the teachings of the following are incorporated herein by reference: U.S. Pat. Publication 2014/0357999, U.S. Pat. 5,357,580, U.S. Pat. 8,721,549 and U.S. Pat. Publication 2012/0136252.

In some embodiments of the invention, scanning of the region of interest comprising a vascular feature may include the steps of imaging a vessel in brightness mode (B-mode), then switching to Doppler signal mode (preferably power Doppler or color Doppler). Within the scope of the invention, it is preferred to preserve the features of the B-mode images by applying appropriate filters thereto, prior to switching to the selected Doppler-mode. For example, this can be achieved by reducing the noise levels (for example, Salt and Pepper Noise (impulse or spike noise), Poisson noise (shot noise), Gaussian or amplifier noise and Speckle Noise). This reduction may be achieved by use of one or more of the following non-limiting filter types: Gaussian filter, bilateral filter, Order statistic filter, Mean filter and Laplacian filter.

Within the scope of the invention, signal processing methods such as wall filters to remove/reduce flash artifacts may be selectively applied to ultrasound images to filter out all frequency shifts that fall below a selected threshold, with the intent of eliminating the lowest Doppler shifts that usually result from vessel (vascular) wall motion and motion in the surrounding solid tissues. These shifts are referred to as noise, clutter, or motion artifacts and are characterized by a low frequency and a high intensity and/or high amplitude. However, an ultrasound scanner and processor may not be able to distinguish between low-frequency Doppler shifts originating from slow-moving blood and those originating from tissue movement. Consequently, both of these low-frequency shifts may be improved or removed when a high filter setting is selected. To avoid a loss of signal related to slow flow, it is preferred that wall filter settings should be kept at the lowest possible setting. Within the scope of the invention, ultrasound scanners may comprise “auto-scan” control functions that automatically adjusts settings (including signal processing filters) according to a selected application.

There are a variety of techniques known and employed in the art to apply such wall filters to ultrasound images. Without limiting the generality of the foregoing, the teachings of the following are incorporated herein by reference: U.S. Pat. 6,760,486.

As such, within the scope of the invention, additional basic parameters for the B-mode (grayscale) examination may preferably be optimized, not only for higher-quality images but also to facilitate the subsequent Doppler component of the method, including the Doppler signal preservation and the depth analysis module. These basic parameters may comprise (a) the location and number of focal zones, (b) the depth of field for the specific vascular feature or ROI being imaged, (c) the two-dimensional (2D) gain setting, (d) the scan orientation, (e) the image zoom settings, and, where possible and depending on the equipment being used, (f) the presets for the specific transducer being used and the type of study being performed. Because color Doppler flow data are superimposed on the 2D image, a high 2D gain setting suppresses color information and a low setting highlights color information. The frame rate varies inversely with the depth of field: Sampling from a deeper segment slows the frame rate.

Prior to commencing the Doppler component of the depth analysis module, a color box may be placed manually over a vascular feature or region of interest or placed over a vascular feature or region of interest, optimally using an artificial intelligence model. Without limiting the generality of the foregoing, the teachings of the following are incorporated herein by reference for AI placement of a color box: U.S. Pat. Publication 2022/0061810.

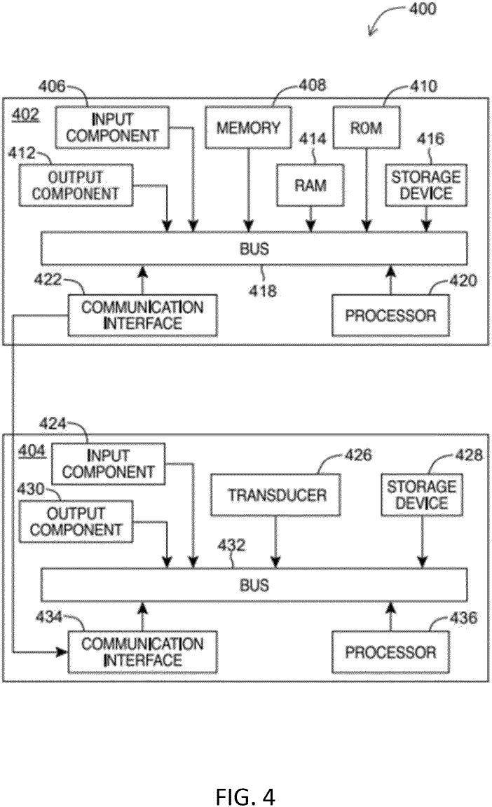

The present invention comprises a depth analysis module. Ultrasound imaging is done using pulse-echo techniques. An ultrasonic transducer is placed in contact with the skin and an ultrasound transducer repeatedly emits brief pulses of sound at a fixed rate, called the pulse repetition frequency, or PRF. After transmitting each pulse, the ultrasound transducer waits for echoes from interfaces along the sound beam path. Echo signals picked up by the transducer are amplified and processed into a format suitable for display, such as on an display unit 402 (FIG. 4) which may comprise an output component 412, such as a display screen, which displays images based on image data acquired by ultrasound acquisition unit 404. The distance to a reflector is determined from the arrival time of its echo. Thus,

$$d = \frac{c T}{2}$$

where d is the depth of the interface, T is the echo arrival time, and c is the speed of sound in the tissue. The factor 2 accounts for the round-trip journey of the sound pulse and echo. Equation 1 is called the range equation in ultrasound imaging. A speed of sound of 1540 m/sec is assumed in most scanners when calculating and displaying reflector depths from echo arrival times. The corresponding echo arrival time is 13 µs/cm of the distance from the transducer to the reflector. By this means and within the scope of the depth analysis module of the invention, T is the echo arrival time of the preserved Doppler signal, whereby d is then automatically calculated and indicated or displayed for a user.

The present invention addresses a critical issue of ascertaining location and depth of one or more vascular features in a fully automated manner such that once a user switches to Doppler mode, the vascular feature depth measurement is immediately made available to a user. There is no need to freeze an image and manually move calipers in order to ascertain a depth value. As such, the human errors associated with improper caliper placement are completely removed and there is an increased time efficiency for operators during medical procedures.

In one aspect of the invention, one or more depth measurements are displayed visually on an interface, such as an interface on a multi-purpose electronic device. This visual display may be, for example the actual depth number encircled or in a prominent, easy to view area of the interface. In another aspect of the invention, the one or more depth measurements may be conveyed to a user audibly. In using presets or other AI enhancement modules, such an interface may also convey to a user, visually or audibly, the identity of one or more vascular features being scanned and for which depths have been ascertained.

In one aspect of the invention, the depth analysis module identifies and calculates a depth measurement of a shallowest vascular vessel (i.e., the shallowest return signal, of the preserved Doppler signals) within the scanned region or region of interest. In another aspect of the invention, the depth analysis module provides and conveys to a user multiple outputs of vascular vessel depths (multiple return signal depths) based on each signal cluster to reduce the dependency on reporting the shallowest depth. This is useful for a user in scanning a region populated with a plurality of vascular features, some shallower and some deeper. In this way, the depth analysis module of the invention may annotate the depth of each of a plurality of vascular features/each flow, based upon a plurality of returned, preserved Doppler signals, as a scanner moves across a scanned region.

In another aspect of the invention, instead of a user identifying a region of interest, there is provided a full screen in a background module (not viewable to a user) on which one or more vascular features are identified and depths calculated in accordance with the present invention. Frames rates may be optimized by one or more means, as Doppler signal acquisitions are slower. For example, line densities may be reduced, as compared to generally accepted line densities and/or ensemble lengths may be reduced (i.e., # of pulses per line that are actually used in the Doppler analysis), as compared to generally accepted ensemble lengths.

In another aspect of the invention, the depth analysis module, employing preserved returned Doppler signals to calculate the depth of one ore more vascular features may be combined with needle enhance/detection technology to concurrently compute and output to a user interface a distance between a tip of a needle and the vessel feature. For example, methods of identify a needle include teachings in U.S. Pat. 10,102,452, the contents of which are incorporated here by reference.

It is intended that method and system of the present invention has wide application in a variety of therapies, procedures, and treatments. Without limiting the generality of the foregoing, the method and system of the present invention may be used in intervention procedures (e.g., nerve blocks, vascular access), wherein needles are used for administration of medicine or evacuation of fluid contents. In the case of nerve blocks it is desirous to avoid all vascular vessels even though many nerves are very closely associated with blood vessels. The method and system of the present invention may be used in a variety of aesthetic and cosmetic procedures are based around injectables wherein avoiding vascular vessels can be a matter of life and death. The method and system of the present invention may be used in surgical procedures such as the Brazilian Butt Lift (BBL) wherein fat is injected into the gluteus region and avoiding vascular features is essential to avoid fat being erroneously deposited therein. The method and system of the present invention provides an means for vascular vessel avoidance, without the need for a user to freeze an ultrasound image and employ calipers for the determination of depth measurements.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

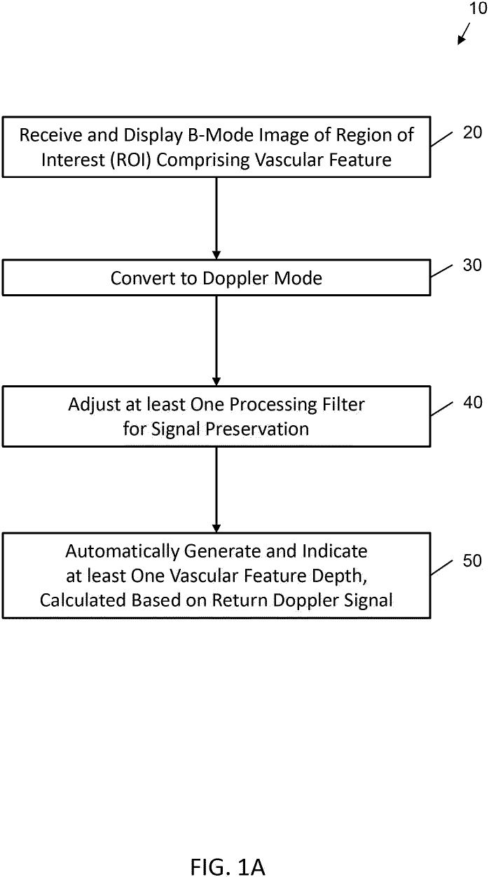

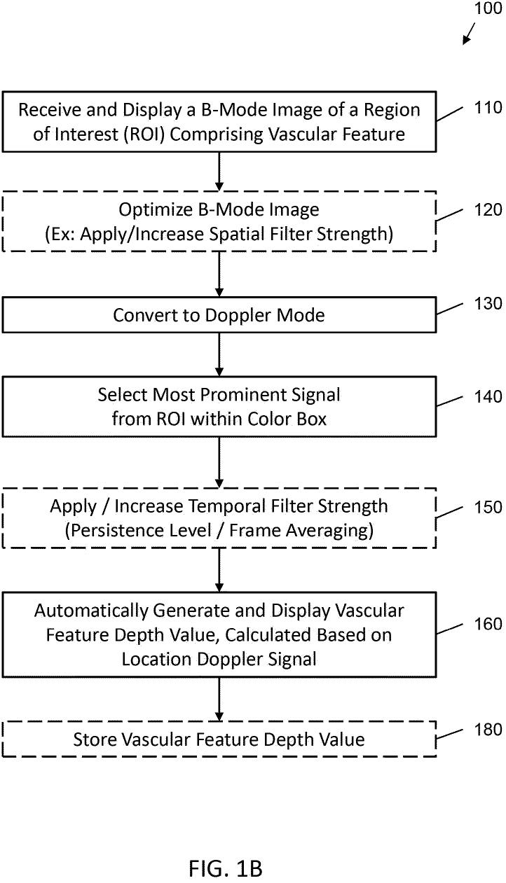

Referring to FIG. 1A, shown there generally as 10 is a flowchart diagram showing steps of a method of automatically determining a depth measurement on vascular feature within an ultrasound image displayed on a device, in accordance with at least one embodiment of the present invention. The various acts of FIG. 1A may generally be performed on a multi-use display device that is operatively connected to an ultrasound scanner. The method may first begin at act 20 by receiving and displaying B-mode images, on the display device, such images comprising a vascular feature within a region of interest (ROI). It is desired to know the depth of that vascular feature without the requirement to freeze an image and employ calipers, such steps being time consuming and prone to human error. At act 30, an operator engages Doppler mode. At act 40, at least one image processing filter is automatically adjusted to create a preserved Doppler signal. For example, this may be achieved by applying/increasing temporal filter strength and/or applying/increasing persistence level. At act 50, a depth analysis module automatically generates and conveys to a user, the depth of the vascular depth feature based on the return of the preserved Doppler mode signal. In this way, by a user simply scanning over a region of interest and switching to a Doppler mode, a depth measurement is automatically displayed or conveyed to user without the requirement for freezing an image and caliper placements.

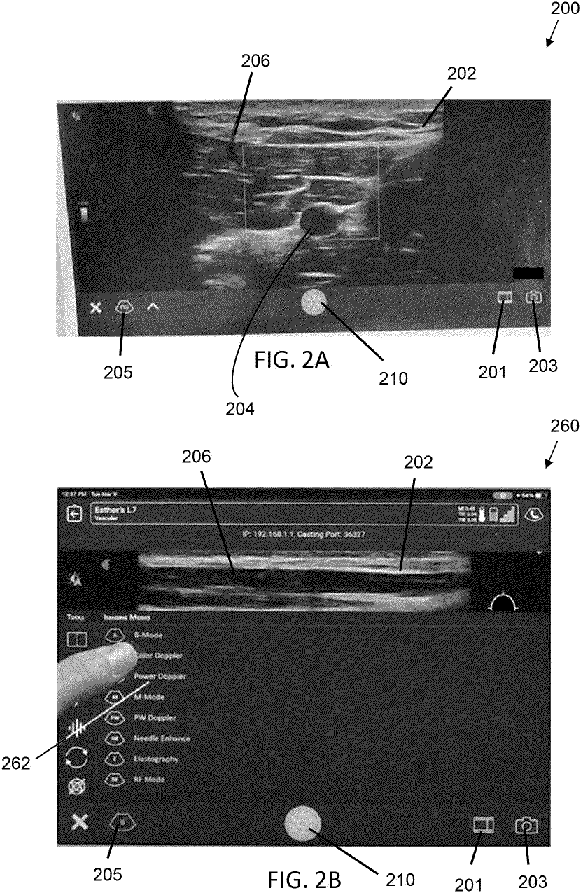

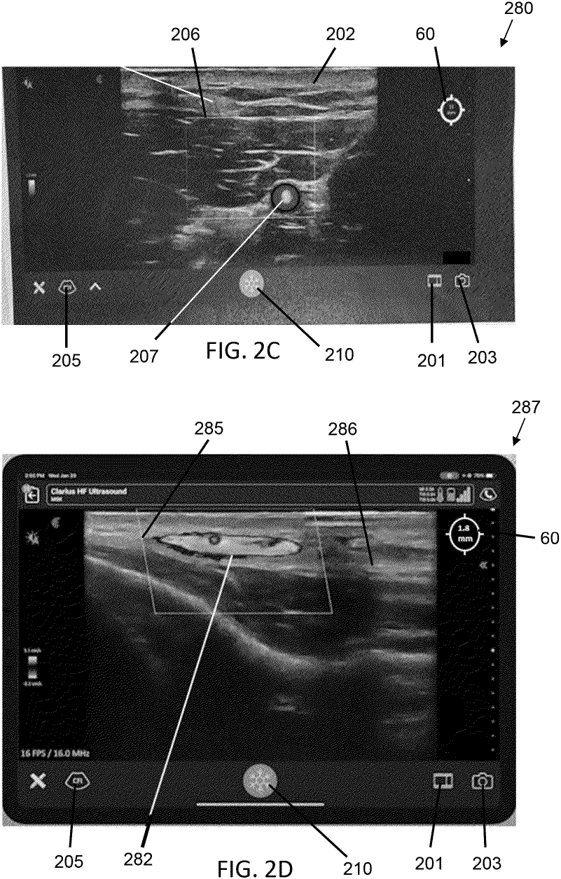

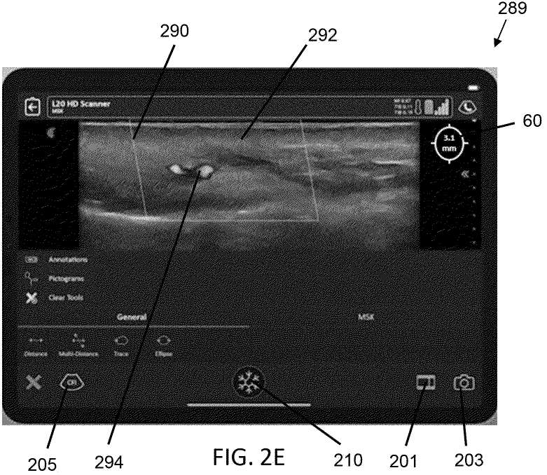

Referring to FIG. 1B, shown there generally as 100 is a flowchart diagram showing steps of a method of automatically determining a depth measurement on vascular feature within an ultrasound image displayed on a touchscreen device, in accordance with at least one embodiment of the present invention. The various acts of FIG. 1B may generally be performed on a multi-use touchscreen display device that is operatively connected to an ultrasound scanner. In discussing the method of FIGS. 1A and 1B, reference will simultaneously be made to FIGS. 2A-2B-2C, which illustrates generally as 200 and 260 and 280, a series of images resulting from performing acts of the method of FIGS. 1A and 1B on an ultrasound image, in accordance with at least one embodiment.

FIGS. 2A-2B-2C show the application of the method of FIGS. 1A and 1B on an example ultrasound image of an artery. The sequence of frames A-C show the results after successive acts of the methods described herein. The example user interface is for an ultrasound imaging scanner that can connect wirelessly to a multi-use display device (e.g., an electronic display unit 402 described in FIG. 4 below). In various embodiments, the device may be a smartphone or a tablet computer. This multi-use display device may also be referred to as a “touchscreen device” herein.

Referring back to FIG. 1B, at act 110 (10 in FIG. 1A), the method may first begin by receiving and displaying B-mode images, on the touchscreen device, such images comprising a vascular feature within a ROI. At act 120, such B-mode images may (optionally) be optimized, for example by increasing spatial filter strength which serves to reduce small noise and inconsequential smaller vessels. In FIG. 2A a B-mode image 202 of an artery 204 is shown, with a broader delineated ROI 206. At act 130 (30 in FIG. 1A), an operator engages Doppler mode (ex: from a selection within a drop-down screen shown in FIG. 2B). In FIG. 2B, the Doppler choice is Power Doppler referenced as numeral 262 and a user is shown to be selecting that mode from a drop-down menu screen. Turning back to FIG. 1B, at act 140, a user may select a most prominent Doppler signal for subsequent steps which includes act 150 (40 in FIG. 1A), optionally applying/increasing temporal filter strength/increase persistence level, thereby created a preserved Doppler signal. At act 160 (50 in FIG. 1A), the depth of the vascular depth feature (based on the the preserved Doppler mode signal) is automatically calculated (as set out above) and conveyed to a user. In other words, a processor uses the data acquired from the Doppler signal to accurately and efficiently create a depth value, which (in this example) is automatically displayed on interface on visual indicator (see visual indicator 60, in each Figure). Referring to FIG. 2C, such an automatic depth display is illustrated as visual indicator 60 (top right of the screen). Optionally at act 180, depth values may be stored for later retrieval and use. In either event, an operator can employ the easy to view and accurate depth values to inject above or below the vascular feature, to avoid the complications noted above. In FIG. 2C, artery/vascular feature is shown as 207 and color box/region of interest as 206.

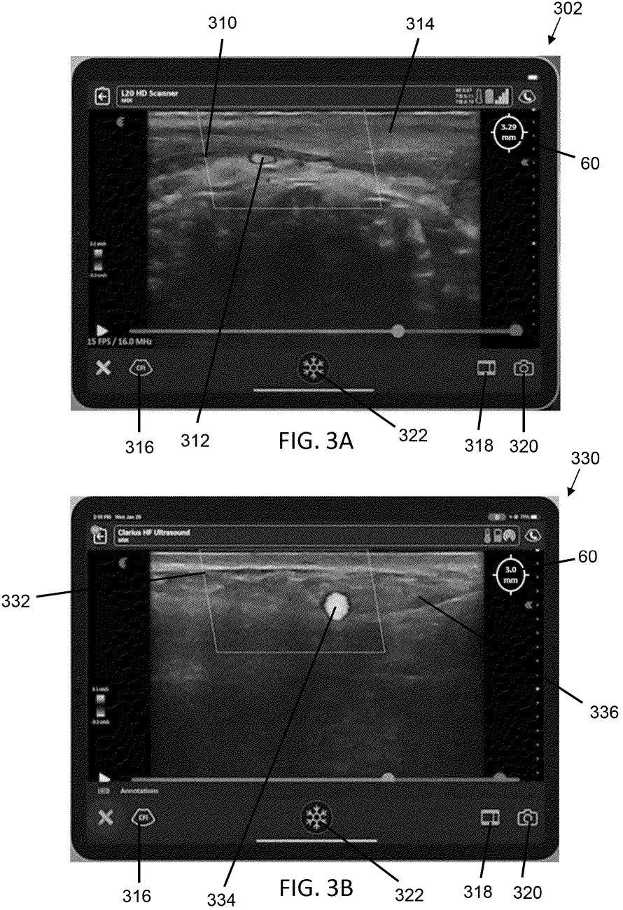

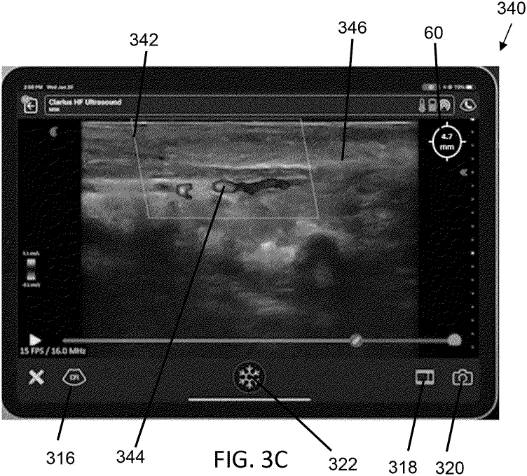

FIGS. 2D, 2E and FIGS. 3A-3C illustrate the equivalent interface for various other vascular featurres, showing Doppler images and each respective depth on the depth indicator icon. In all of FIGS. 2A-2E, mode indicator is shown as 205, video icon as 201, camera (snapshot) icon as 203 and freeze button shown as 210. In FIG. 2D, generally shown at 287 is interface of a multi-purpose device, displaying a vascular feature which is superficial temporal artery 282 on image 286 within color box 285, with an automatically calculated depth of 1.8 mm shown at icon 60 in the top right corner. In FIG. 2E, generally shown at 289 is interface of a multi-purpose device, displaying a vascular feature which is angular artery 294 on image 292 within color box 290, with an automatically calculated depth of 3.1 mm shown at icon 60 in the top right corner.

In FIG. 3A, generally shown at 302 is interface of a multi-purpose device, displaying a vascular feature which is the inferior labial artery 312 on image 314 within color box 310, with an automatically calculated depth of 3.29 mm shown at icon 60 in the top right corner. In FIG. 3B, generally shown at 330 is interface of a multi-purpose device, displaying a vascular feature which is the facial artery 334 on image 336 within color box 332, with an automatically calculated depth of 3.0 mm shown at icon 60 in the top right corner. In FIG. 3C, generally shown at 340 is interface of a multi-purpose device, displaying a vascular feature which is the superior labial artery 344 on image 346 within color box 342, with an automatically calculated depth of 4.7 mm shown at icon 60 in the top right corner. In all of FIGS. 3A-3C, mode indicator is shown as 316, video icon as 318, camera (snapshot) icon as 320 and freeze button shown as 322.

Referring to FIG. 4, shown there generally as 400 is a functional block diagram of an ultrasound system and/or apparatus, in accordance with at least one embodiment of the present invention. For example, one or more components of the ultrasound imaging system 400 may be configured to perform the methods of FIGS. 1 to perform a measurement on an ultrasound image displayed on a touchscreen device.

Ultrasound imaging system 400 may include an ultrasound acquisition unit 404 configured to transmit ultrasound energy to a target object, receive ultrasound energy reflected from the target object, and generate ultrasound image data based on the reflected ultrasound energy. The ultrasound acquisition unit 404 may include a transducer 426 which converts electric current into ultrasound energy and vice versa. Transducer 426 may transmit ultrasound energy to the target object which echoes off the tissue. The echoes may be detected by a sensor in transducer 426 and relayed through a bus 432 to a processor 436. Processor 436 may interpret and process the echoes to generate image data of the scanned tissue. In some embodiments, the ultrasound acquisition unit 404 (or various components thereof) may be provided as a handheld ultrasound probe or scanner that is in communication with other components of the ultrasound imaging system 400. For example, the handheld probe may include the transducer 426 of ultrasound acquisition unit 404. Ultrasound acquisition unit 404 may also include storage device 428 (e.g., a computer readable medium, coupled to and accessible by bus 432) for storing software or firmware instructions, configuration settings (e.g., sequence tables), and/or ultrasound image data.

Although not illustrated, as will be apparent to one of skill in the art, the ultrasound imaging system 400 may include other components for acquiring, processing and/or displaying ultrasound image data. These include, but are not limited to: a scan generator, transmit beamformer, pulse generator, amplifier, analogue to digital converter (ADC), receive beamformer, signal processor, data compressor, wireless transceiver and/or image processor. Each of these may be components of ultrasound acquisition unit 404 and/or electronic display unit 402 (described below).

Ultrasound imaging system 400 may include an electronic display unit 402 which is in communication with ultrasound acquisition unit 404 via communication interfaces 422 / 434. In various embodiments, communication interfaces 422 / 434 may allow for wired or wireless connectivity (e.g., via Wi-Fi™ and/or Bluetooth™) between the electronic display unit 402 and the ultrasound acquisition unit 404. Electronic display unit 402 may work in conjunction with ultrasound acquisition unit 404 to control the operation of ultrasound acquisition unit 404 and display the images acquired by the ultrasound acquisition unit 404. An ultrasound operator may interact with the user interface provided by display unit 402 to send control commands to the ultrasound acquisition unit 404 (e.g., to change presets). The electronic display unit 402 may have been referred to as a multi-use display device, a touchscreen device, and/or a mobile device above. In various embodiments, the electronic display unit 402 may be a portable device, which may include a mobile device (e.g. smartphone), tablet, laptop, or other suitable device incorporating a display and a processor and capable of accepting input from a user and processing and relaying the input to control the operation of the ultrasound acquisition unit 404 as described herein.

Each of ultrasound acquisition unit 404 and display unit 402 may have one or more input components 424, 406 and/or one or more output components 430, 412. In the FIG. 4 embodiment, ultrasound acquisition unit 404 may include an input component 424 which is configured to accept input from the user (e.g., a user-programmable button for adjusting imaging parameters; and/or circuitry to turn on the ultrasound acquisition unit 404 and/or control the connection of the ultrasound acquisition unit 404 to the electronic display unit 402). For example, in some embodiments, ultrasound acquisition unit 404 may also include an output component 430, such as a LED indicator light which can output the status of the ultrasound acquisition unit 404.

In the FIG. 4 embodiment, display unit 402 may include an input component 406 configured to accept input from the user. Certain input received at input component 406 may be relayed to ultrasound acquisition unit 404 to control the operation of ultrasound acquisition unit 404. Display unit 402 may also include an output component 412, such as a display screen, which displays images based on image data acquired by ultrasound acquisition unit 404. In particular embodiments, display unit 402′s input component 406 may include a touch interface layered on top of the display screen of the output component 412, so as to provide a touchscreen interface. Electronic display unit 402 may also include memory 408, Random Access Memory (RAM) 414, Read Only Memory (ROM) 410, and persistent storage device 416, which may all be connected to bus 418 to allow for communication therebetween and with processor 420. Any number of these memory elements may store software or firmware that may be accessed and executed by processor 420 to perform the acts of the methods described herein (e.g., so that the processor 420 is configured to perform the methods described herein of performing a measurement on an ultrasound image displayed on a touchscreen device).

In various embodiments, at least a portion of the processing of the image data corresponding to the reflected ultrasound energy detected by the transducer 426 may be performed by one or more of processors internal to the ultrasound acquisition unit 404 (such as by the processor 436) and/or by processors external to the ultrasound acquisition unit 404 (such as the processor 420 of electronic display unit 402).

Scan conversion is a process that converts image data to allow it to be displayed in a form that is more suitable for human visual consumption. For example, this may involve converting the image data from the data space (e.g. polar coordinate form) to the display space (e.g. Cartesian coordinate form). In an example embodiment, the ultrasound acquisition unit 404 may provide pre-scan-converted data to the electronic display unit 402, and the electronic display unit 402 may proceed to scan convert the data. The methods described herein then generally be performed on the post-scan-converted data at display unit 402 with a touchscreen device.

In some embodiments, the ultrasound acquisition unit 404 may have a lightweight, portable design and construction (e.g., when it is a handheld probe). In particular embodiments, the handheld probe may have a mass that is less than approximately 1 kg (2 lbs).

In some embodiments, all the input controls and display screen necessary for the operation of the ultrasound imaging system 400 may be provided by input and output components 406, 412 of the display unit 402. In such cases input and output components 424, 430 of ultrasound acquisition unit 404 may be optional and/or omitted. As noted, in certain embodiments, the ultrasound acquisition unit 404 may be a handheld probe (e.g., including transducer 426) which is in communication with the display unit 402 over the communications interfaces 422/434 to facilitate operation of the ultrasound acquisition unit 404 and processing and display of ultrasound images.

In some embodiments, a display device may host a screen and may include a processor, which may be connected to a non-transitory computer readable memory storing computer readable instructions, which, when executed by the processor, cause the display device to provide one or more of the functions of the system of the invention. Such functions may be, for example, the receiving of ultrasound data that may or may not be pre-processed; scan conversion of received ultrasound data into an ultrasound image; processing of ultrasound data in image data frames; the display of a user interface; the control of the scanner; the display of an ultrasound image on the screen; the processing of a switch from one signal acquiring mode to another (i.e., a Doppler mode), the placement of a color box, the application of one or more signal processing filters to create preserved Doppler signals, processing preserved Doppler signals in a depth analysis module and indicating to a user depth measurements of one ore more vascular features.

In some embodiments, the output component 430 of ultrasound acquisition unit 404 may include a display screen, which can be configured to display or otherwise output the images acquired by ultrasound acquisition unit 404 (in addition to or alternative to displaying such images on the display unit 402).

While a number of exemplary aspects and embodiments have been discussed above, those of skill in the art will recognize that may be certain modifications, permutations, additions and sub-combinations thereof. While the above description contains many details of example embodiments, these should not be construed as essential limitations on the scope of any embodiment. Many other ramifications and variations are possible within the teachings of the

### C. CLAIM SUPPORT

In a first broad aspect of the present disclosure, there is provided a method for automatically determining a depth of a vascular feature on an ultrasound image feed, acquired from an ultrasound scanner, the method comprising: displaying, on a screen that is communicatively connected to the ultrasound scanner, the ultrasound image feed comprising ultrasound image frames of a region of interest comprising the vascular feature; activating a Doppler mode of the ultrasound scanner, in which the ultrasound scanner obtains a Doppler-mode ultrasound signal corresponding to the region of interest comprising the vascular feature; applying at least one image processing filter to preserve the Doppler-mode ultrasound signal (the “preserved Doppler-mode signal”); generating from the preserved Doppler-mode signal of the vascular feature as returned to the ultrasound scanner, the depth of the vascular feature; and indicating depth of the vascular feature to a user of ultrasound scanner.

In some embodiments, the at least one image processing filter is a temporal filter.

In some embodiments, the temporal filter is a flash removal filter which preserves the Doppler-mode ultrasound signal by increasing a number of ultrasound image frames of the region of interest from the ultrasound image feed.

In some embodiments, the temporal filter is an adaptive persistence filter which is increased to preserve the Doppler-mode ultrasound signal by averaging a plurality of ultrasound image frames of the region of interest from the ultrasound mage feed.

In some embodiments, an additional step is provided of optimizing images by applying a wall filter prior to activating the Doppler mode of the ultrasound scanner.

In some embodiments, an additional step is provided, after activating the Doppler mode of the ultrasound scanner, of selecting a prominent Doppler-mode ultrasound signal corresponding to the region of interest comprising the vascular feature.

In some embodiments, an additional step is provided, after activating the Doppler mode of the ultrasound scanner, of placing a color box place on the region of interest.

In some embodiments, the steps of applying at least one image processing filter, generating from the preserved Doppler-mode signal the depth of the vascular feature and displaying depth of the vascular feature on the screen occur in real time and without additional user inputs.

In some embodiments, the screen is within a multi-purpose electronic device which is communicatively coupled with the ultrasound scanner and the step of indicating depth of the vascular feature to a user of ultrasound scanner is via at least one of a visual or an audio signal.

In some embodiments, the vascular feature is any tissue through which blood flows and for which an automatic depth measurement from a skin surface is instructive for the purposes of therapy, procedures, diagnosis, or treatment.

In a second broad aspect of the present disclosure, there is provided an ultrasound imaging system for automatically determining a depth of a vascular feature on an ultrasound image feed, comprising: an ultrasound scanner configured to acquire a plurality of new ultrasound frames; processor that is communicatively connected to the ultrasound scanner and configured to: display, on a screen that is communicatively connected to the ultrasound scanner, the ultrasound image feed comprising ultrasound image frames of a region of interest comprising the vascular feature; activate a Doppler mode of the ultrasound scanner, in which the ultrasound scanner obtains a Doppler-mode ultrasound signal corresponding to the region of interest comprising the vascular feature; apply at least one image processing filter to preserve the Doppler-mode ultrasound signal (the “preserved Doppler-mode signal”); generate from the preserved Doppler-mode signal of the vascular feature as returned to the ultrasound scanner, the depth of the vascular feature; and a display device configured to indicate the depth of the vascular feature to a system user.

In some embodiments, the at least one image processing filter is a temporal filter.

In some embodiments, the temporal filter is a flash removal filter which preserves the Doppler-mode ultrasound signal by increasing a number of post scan converted ultrasound image frames of the region of interest from the ultrasound image feed.

In some embodiments, the temporal filter is an adaptive persistence filter which is increased to preserve the Doppler-mode ultrasound signal by averaging a plurality of post scan converted ultrasound image frames of the region of interest from the ultrasound mage feed.

In some embodiments, the processor is additionally configured to optimize images captured in 2D-mode by applying a wall filter prior to switching the ultrasound scanner from the 2D-mode to Doppler mode.

In some embodiments, the processor is additionally configured to, after switching the ultrasound scanner from the 2D-mode to Doppler mode, select a prominent Doppler-mode ultrasound signal corresponding to the region of interest comprising the vascular feature.

In some embodiments, the processor is additionally configured to, after switching the ultrasound scanner from the 2D-mode to Doppler mode, place a color box place on the region of interest.

In some embodiments, the display device is a multi-purpose electronic device which is communicatively coupled with the ultrasound scanner and indicating depth of the vascular feature to a user of ultrasound scanner is via at least one of a visual or an audio signal.

In some embodiments, the processor applies at least one image processing filter, generates from the preserved Doppler-mode signal the depth of the vascular feature and indicates the depth of the vascular feature in real time and without additional user inputs.

In a third broad aspect of the present disclosure, there is provided a computer readable medium storing instruction for execution by a processor communicatively coupled with an ultrasound scanner, within an ultrasound imaging system, wherein when the instructions are executed by the processor, it is configured to: display, on a screen that is communicatively connected to the ultrasound scanner, an ultrasound image feed comprising ultrasound image frames of a region of interest comprising a vascular feature; activate a Doppler mode of the ultrasound scanner, in which the ultrasound scanner obtains a Doppler-mode ultrasound signal corresponding to the region of interest comprising the vascular feature; apply at least one image processing filter to preserve the Doppler-mode ultrasound signal (the “preserved Doppler-mode signal”); generate from the preserved Doppler-mode signal of the vascular feature as returned to the ultrasound scanner, the depth of the vascular feature; indicate depth of the vascular feature to a user of the ultrasound imaging system.

### D. Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

To aid the Patent Office and any readers of any patent issued on this application in interpreting the claims appended hereto, applicant wishes to note that they do not intend any of the appended claims or claim elements to invoke 35 U.S.C. 112(f) unless the words “means for” or “step for” are explicitly used in the particular claim.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

What is claimed is:

1. A method for automatically determining a depth of a vascular feature on an ultrasound image feed, acquired from an ultrasound scanner, the method comprising:
displaying, on a screen that is communicatively connected to the ultrasound scanner, the ultrasound image feed comprising ultrasound image frames of a region of interest comprising the vascular feature;
activating a Doppler mode of the ultrasound scanner, in which the ultrasound scanner obtains a Doppler-mode ultrasound signal corresponding to the region of interest comprising the vascular feature;
applying at least one image processing filter to preserve the Doppler-mode ultrasound signal (the “preserved Doppler-mode signal”);
generating from the preserved Doppler-mode signal of the vascular feature as returned to the ultrasound scanner, the depth of the vascular feature; and
indicating depth of the vascular feature to a user of ultrasound scanner.

2. The method of claim 1 wherein the at least one image processing filter is a temporal filter.

3. The method of claim 2 wherein the temporal filter is a flash removal filter which preserves the Doppler-mode ultrasound signal by increasing a number of ultrasound image frames of the region of interest from the ultrasound image feed.

4. The method of claim 1 wherein the temporal filter is an adaptive persistence filter which is increased to preserve the Doppler-mode ultrasound signal by averaging a plurality of ultrasound image frames of the region of interest from the ultrasound mage feed.

5. The method of claim 1 comprising an additional step of optimizing images by applying a wall filter prior to activating the Doppler mode of the ultrasound scanner.

6. The method of claim 1 comprising an additional step, after activating the Doppler mode of the ultrasound scanner, of selecting a prominent Doppler-mode ultrasound signal corresponding to the region of interest comprising the vascular feature.

7. The method of claim 1 comprising an additional step, after activating the Doppler mode of the ultrasound scanner, of placing a color box place on the region of interest.

8. The method of claim 1 wherein the steps of applying at least one image processing filter, generating from the preserved Doppler-mode signal the depth of the vascular feature and displaying depth of the vascular feature on the screen occur in real time and without additional user inputs.

9. The method of claim 1, wherein the screen is within a multi-purpose electronic device which is communicatively coupled with the ultrasound scanner and the step of indicating depth of the vascular feature to a user of ultrasound scanner is via at least one of a visual or an audio signal.

10. The method of claim 1 wherein the vascular feature is any tissue through which blood flows and for which an automatic depth measurement from a skin surface is instructive for the purposes of therapy, procedures, diagnosis, or treatment.

11. An ultrasound imaging system for automatically determining a depth of a vascular feature on an ultrasound image feed, comprising:
an ultrasound scanner configured to acquire a plurality of new ultrasound frames;
processor that is communicatively connected to the ultrasound scanner and configured to:
display, on a screen that is communicatively connected to the ultrasound scanner, the ultrasound image feed comprising ultrasound image frames of a region of interest comprising the vascular feature;
activate a Doppler mode of the ultrasound scanner, in which the ultrasound scanner obtains a Doppler-mode ultrasound signal corresponding to the region of interest comprising the vascular feature;
apply at least one image processing filter to preserve the Doppler-mode ultrasound signal (the “preserved Doppler-mode signal”);
generate from the preserved Doppler-mode signal of the vascular feature as returned to the ultrasound scanner, the depth of the vascular feature;
a display device configured to indicate the depth of the vascular feature to a system user.

12. The ultrasound imaging system of claim 11 wherein the at least one image processing filter is a temporal filter.

13. The ultrasound imaging system of claim 12 wherein the temporal filter is a flash removal filter which preserves the Doppler-mode ultrasound signal by increasing a number of post scan converted ultrasound image frames of the region of interest from the ultrasound image feed.

14. The ultrasound imaging system of claim 11 wherein the temporal filter is an adaptive persistence filter which is increased to preserve the Doppler-mode ultrasound signal by averaging a plurality of post scan converted ultrasound image frames of the region of interest from the ultrasound mage feed.

15. The ultrasound imaging system of claim 11 wherein processor is additionally configured to optimize images captured in 2D-mode by applying a wall filter prior to switching the ultrasound scanner from the 2D-mode to Doppler mode.

16. The ultrasound imaging system of claim 11 wherein processor is additionally configured to, after switching the ultrasound scanner from the 2D-mode to Doppler mode, select a prominent Doppler-mode ultrasound signal corresponding to the region of interest comprising the vascular feature.

17. The ultrasound imaging system of claim 11 wherein processor is additionally configured to, after switching the ultrasound scanner from the 2D-mode to Doppler mode, place a color box place on the region of interest.

18. The ultrasound imaging system of claim 11, wherein the display device is a multi-purpose electronic device which is communicatively coupled with the ultrasound scanner and indicating depth of the vascular feature to a user of ultrasound scanner is via at least one of a visual or an audio signal.

19. The ultrasound imaging system of claim 11 wherein the processor applies at least one image processing filter, generates from the preserved Doppler-mode signal the depth of the vascular feature and indicates the depth of the vascular feature in real time and without additional user inputs.

20. A computer readable medium storing instruction for execution by a processor communicatively coupled with an ultrasound scanner, within an ultrasound imaging system, wherein when the instructions are executed by the processor, it is configured to:
display, on a screen that is communicatively connected to the ultrasound scanner, an ultrasound image feed comprising ultrasound image frames of a region of interest comprising a vascular feature;
activate a Doppler mode of the ultrasound scanner, in which the ultrasound scanner obtains a Doppler-mode ultrasound signal corresponding to the region of interest comprising the vascular feature;
apply at least one image processing filter to preserve the Doppler-mode ultrasound signal (the “preserved Doppler-mode signal”);
generate from the preserved Doppler-mode signal of the vascular feature as returned to the ultrasound scanner, the depth of the vascular feature;
indicate depth of the vascular feature to a user of the ultrasound imaging system.
